(19) United States
(12) Patent Application Publication
(10) Pub. No.: US 2025/0066743 A1
(43) Pub. Date: Feb. 27, 2025

(54) METHODS AND COMPOSITIONS FOR PURIFYING ADENO ASSOCIATED VIRUS PARTICLES

(71) Applicant: MERCK PATENT GMBH, Darmstadt (DE)

(72) Inventors: Bianca EDELMANN, Darmstadt (DE); Helena MARIE, Darmstadt (DE); Oliver RAMMO, Darmstadt (DE); Karen KLEWER, Darmstadt (DE)

(73) Assignee: MERCK PATENT GMBH, Darmstadt (DE)

(21) Appl. No.: 18/847,937

(22) PCT Filed: Mar. 15, 2023
(86) PCT No.: PCT/EP2023/056548

§ 371 Date: Sep. 17, 2024

(30) Foreign Application Priority Data
Mar. 18, 2022 (EP) 22162928.0

(51) Int. Cl. C12N 7/00; B01D 15/36; B01D 15/38; B01D 15/42

(52) U.S. Cl. CPC C12N 7/00; B01D 15/362; B01D 15/3847; B01D 15/424; C12N 2750/14151

(57) ABSTRACT

The present invention relates to methods for purifying adeno-associated virus particles by cation exchange mixed mode chromatography.

The present invention relates to methods for purifying adeno-associated virus particles by cation exchange mixed mode chromatography.

Adeno-associated virus (AAV) have been characterized and developed as a potent viral vector to deliver genes in vitro in cultured cells and also in vivo. AAV is meanwhile a leading platform for in vivo delivery of gene therapies. AAV is a small, non-enveloped virus containing a single-stranded DNA genome of approximately 4.7 kb, consisting of two inverted terminal repeats (ITRs) that are capable of forming T-shape secondary structure and acting as origins of genome replication, one rep region that encodes four overlapping replication proteins, Rep78, Rep68, Rep52, and Rep40, and one cap region that encodes three structural proteins, VP1, VP2, and VP3, and an assembly activating protein (AAP). Naturally isolated serotypes 1-9 of the AAV viruses share the genomic structure although these serotypes may display different tissue tropism. As the AAVs seem to be nonpathogenic, show an efficient transduction and a stable expression, they are regarded as being one of the most promising gene delivery vehicles.

AAV vectors can be produced in various cell lines in adherent or suspension cell culture formats using transient transfection or co-infection methods. Depending on specific serotypes and production times, viral particles including full, partial and empty species can be secreted out of cells into culture medium or contained inside cells at various ratios.

Initially, stable AAV producer cells were generated by transfection and selection of human-derived cells, like HeLa or HEK293 cells, with an rAAV transfer vector containing the ITR cassette and a packaging construct containing Rep and Cap. Production of recombinant AAV vectors (rAAV) was then achieved by infection with auxiliary viruses such as adenoviruses (AdV) that provide the helper function. After identification of AdV genes required for AAV vector packaging, a helper virus-free method was established using a duo or triple transfection protocol consisting of two or three plasmids including a constructed helper plasmid instead of an auxiliary virus. This system is widely used in research and drug development. In addition, development of baculovirus expression vectors provides another method to produce rAAV viruses in insect Sf9 cells. These different technologies are shown to be able to produce sufficient quantities of rAAV viruses for use in laboratories and clinical trials.

A cell lysis step is generally required at harvest to release viral particles into the supernatant. For this application, typical cell lysis reagents such as Triton X-100, Tween 20, and NaCl are broadly utilized.

After cell lysis, the AAVs need to be purified. Typical AAV purification processes include clarification, concentration and diafiltration using tangential flow filtration, chromatography purification by using affinity chromatography and ion exchange chromatography. In some processes, ultracentrifugation and gradient ultracentrifugation are used instead of chromatography or in addition to chromatography. Final steps in AAV purification typically involves concentration and diafiltration into suitable excipient buffer composition and sterile filtration.

Although there is an increasing demand for viral vectors as vehicles in gene therapy, current manufacturing processes suffer from purification methods that simultaneously scale to meet the production demands but also reduce the impurity burden. Due to their ability to induce diving and non-diving tissues, patient safety and the control for cell specific applications, adeno-associated virus (rAAV) is an attractive vector in that field. However, producing these viruses while increasing efficiency and lowering manufacturing costs is a difficult task for downstream processing.

A major challenge in current AAV processes is the production of non-genome containing particles. Although their mechanistic of action is not fully understood, empty rAAV capsids are treated as a major process impurity that can hamper safety and efficacy of the final formulated drug. Independent of being produced in mammalian or insect cell lines, resulting rAAV feed streams typically contain a level of 10-90% empty capsids that need to be removed further downstream. A typically downstream scheme consists of two steps; an initial affinity capture step to capture all rAAV particles from the feed stream and remove other process related impurities and a subsequent polish step to separate full from empty rAAV particles. Full capsids have been explored to have higher charge density due to the negatively charged genome that they carry. And even though this negatively charged genome changes the isoelectric point (pI) just slightly (empty: pI=5.9 vs. full: pI=6.3), these attributes are enough that ion exchange chromatography, in particular anion exchange chromatography (AEX) has been widely explored for this type of application (Wang, Dan; Tai, Phillip W. L.; Gao, Guangping (2019): Adeno-associated virus vector as a platform for gene therapy delivery. In: Nature reviews. Drug discovery 18 (5), S. 358-378. DOI: 10.1038/s41573-019-0012-9).

In WO04113494 methods are disclosed for separating empty and full particles by single or multiple anion- and or cation exchange steps.

Nevertheless there is still the need for an efficient and simple method that provides for purification of target rAAVs from process related impurities as well as for separation of empty and full capsids.

The inventors have surprisingly found that mixed mode cation exchange chromatography is especially efficient in purifying rAAVs from process related impurities like host cell proteins. Typically, the empty capsids can also be removed partially or fully in the same chromatography step.

The present invention is thus directed to a method for purifying adeno-associated virus (AAV) particles by contacting a sample comprising said AAV particles with a mixed mode cation exchange chromatography matrix. Said chromatography matrix comprises cation exchange and hydrophobic groups.

In a preferred embodiment the method of the present invention comprises the steps of
a) contacting the sample comprising the AAV particles with a mixed mode cation exchange chromatography matrix
b) optionally washing the chromatography matrix
c) eluting AAV particles which bound to the chromatography matrix in step a) with an elution buffer

In a preferred embodiment, in step c), the elution buffer has a pH higher than the loading buffer. The elution buffer may be applied as step or as gradient.

In another embodiment, the method includes recovering AAV particles which flow through the chromatography matrix without binding to it.

In a preferred embodiment the chromatography matrix is a membrane or a monolith.

In a very preferred embodiment the chromatigraphy matrix is a membrane, especially a hydrogel membrane.

In another preferred embodiment the sample comprises empty and full AAV capsids and the a mixed mode cation exchange chromatography matrix is contacted with the sample under conditions so that the majority of AAV particles, empty and full particles, bind to the chromatography matrix while the majority of process related impurities flow through the matrix.

In another preferred embodiment the elution takes place under such conditions that the majority of empty AAV capsids comes of a mixed mode cation exchange chromatography matrix together with other impurities prior to the majority of full AAV particles.

In another preferred embodiment the AAVs purified with the method of the present invention have a proportion of full to emptyAAVs of more than 70% full AAVs, preferably more than 90%, most preferred more than 99% full AAVs, which means that empty particles are reduced down to below 10%, more preferred down to below 1%.

In a preferred embodiment the sample is a crude lysate.

In another preferred embodiment elution in step c) is performed with a linear pH gradient from a pH between 4 and 6.5 to a pH between 9 and 11 while conductivity is kept at a constant level, preferrably at a constant level between 1 mS/cm and 50 mS/cm, most preferred between 13 and 20 mS/cm. Preferably, the target AAVs are eluted at a pH above 8.0, especially at a pH between 8.5 and 10.5, while empty AAVs as well as process related impurities like HCPs are eluted prior to the target AAVs at a pH below 8.

In a preferred embodiment, in one fraction of AAVs eluted in step c) HCPs are reduced with a log reduction of 2 log or more compared to the sample applied in step a) and the percentage of full AAVs is above 90%.

In an especially preferred embodiment this fraction results from an elution with a linear pH gradient from a pH between 4 and 6.5 to a pH between 9 and 11 while conductivity is kept at a constant level and said fraction is eluted at a pH above 8.0, especially it is the fraction eluted between pH 8.5 and 10.5.

The present invention is further directed to a method for purifying AAV particles from a sample comprising cells enclosing the viral particles by
a) lysing the cells
b) isolating and/or purifying the AAV particles by contacting it with a mixed mode cation exchange chromatography matrix.

In a preferred embodiment, lysis of the cells is done with a detergent selected from the group of alkyldimethylamine oxides and optionally sodium chloride.

In one embodiment, the method of the present invention comprises one or more of the following steps:
Clarification
filtration
dialysis/diafiltration
tangential flow filtration
treatment with nuclease, e.g. RNase and/or DNase
treatment with chloroform
ion exchange chromatography
affinity chromatography
hydrophobic interaction chromatography
centrifugation
PEG precipitation

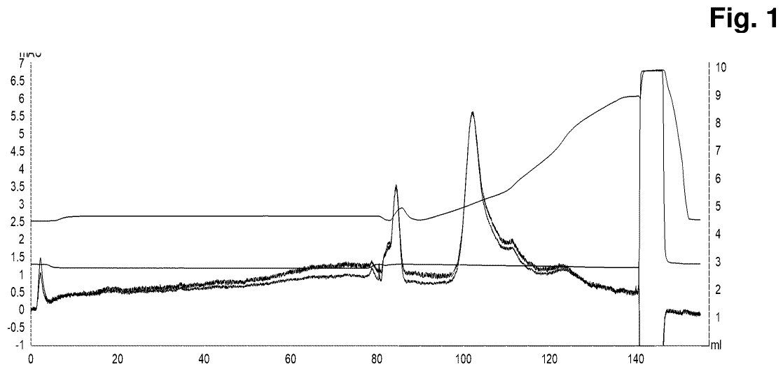

FIG. 1 shows the run on Eshmuno@ CMX column, running on the ÄKTA™ system. (UV-280 nm blue; 260 nm red). During the load no AAV could be detected. In the wash step the first peak occurred and during the elution gradient a second peak could be eluted. The overall recovery of all rAAV2 capsids was 78% and 77% for full rAAV2 capsids, but no enrichment in the elution peak was observed.

Further details can be found in Example 1.

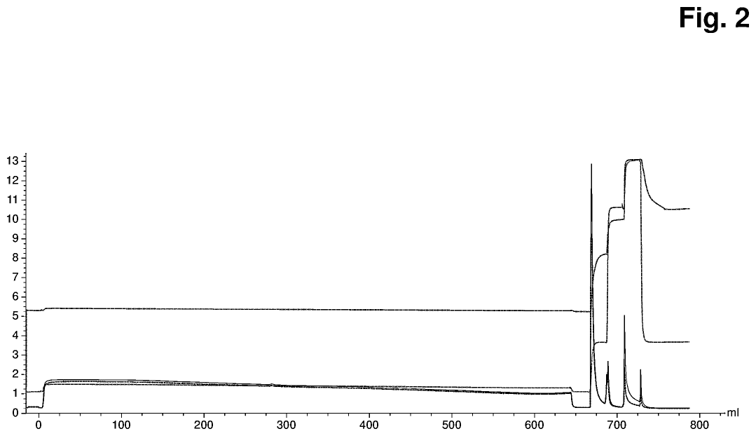

FIG. 2 shows the chromatogram of AAV2 of capture run using a step gradient elution on mixed-mode membrane of 1 mL volume. Equilibration buffer consists of a combination of salts and 120 mM NaCl and pH 5.3, whereby the elution buffer contains 500 mM NaCl and same constituting salts and pH 8.5. Further details can be found in Example 2.

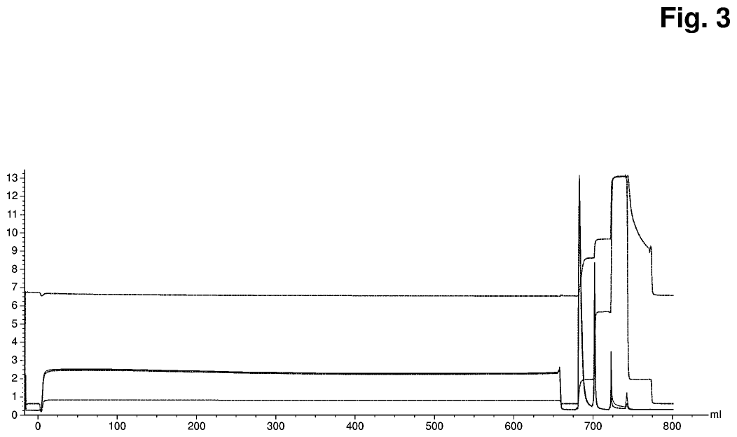

FIG. 3 shows Chromatogram of AAV2 of capture run using a step gradient elution on mixed-mode membrane of 1 mL volume, aiming to improve HCP clearance. Operating conditions: pH 6.5, 120 mM NaCl, whereby the elution buffer contains 500 mM NaCl and same constituting salts and pH 8.5.

Further details can be found in Example 3.

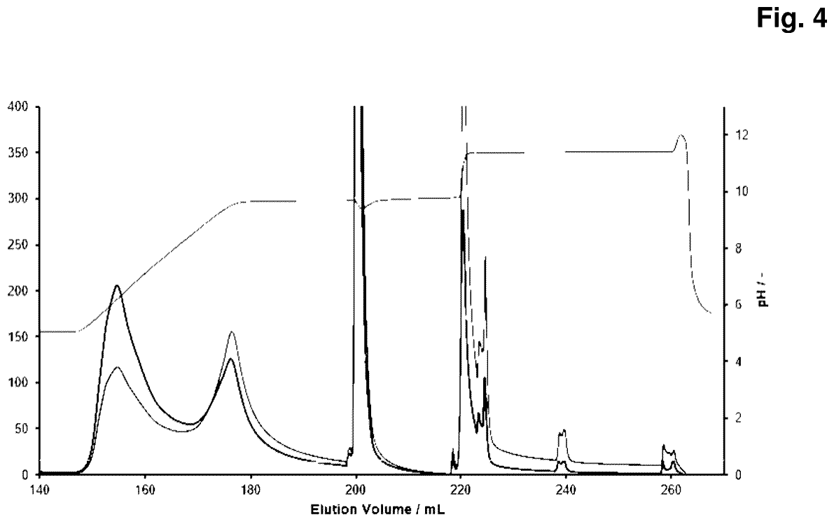

FIG. 4 shows Chromatogram of rAAV2 capture run using a linear gradient elution on mixed-mode membrane of 1 mL volume, aiming to improve HCP clearance and full rAAV2 capsids on step. Binding conditions: pH 5.3, 150 mM NaCl, whereby the elution buffer contains 150 mM NaCl and same constituting salts and pH 10.

Further details can be found in Example 4.

Before describing the present invention in detail, it is to be understood that this invention is not limited to specific compositions or process steps, as such may vary. It must be noted that, as used in this specification and the appended claims, the singular form “a”, “an” and “the” include plural referents unless the context clearly dictates otherwise. Thus, for example, reference to “a ligand” includes a plurality of ligands and reference to “an antibody” includes a plurality of antibodies and the like.

Unless defined otherwise, all technical and scientific terms used herein have the same meaning as commonly understood by one of ordinary skill in the art to which this invention is related. The following terms are defined for purposes of the invention as described herein.

Adeno-associated virus (AAV) is a member of the Parvoviridae family. The AAV genome is composed of a linear single-stranded DNA molecule which contains approximately 4.7 kilobases (kb) and consists of two major open reading frames encoding the non-structural Rep (replication) and structural Cap (capsid) proteins. Flanking the AAV coding regions are two cis-acting inverted terminal repeat (ITR) sequences, approximately 145 nucleotides in length, with interrupted palindromic sequences that can fold into hairpin structures that function as primers during initiation of DNA replication. In addition to their role in DNA replication, the ITR sequences have been shown to be necessary for viral integration, rescue from the host genome, and encapsidation of viral nucleic acid into mature virions (Muzyczka, (1992) Curr. Top. Micro. Immunol. 158:97-129).

Multiple serotypes of AAV exist and offer varied tissue tropism. Known serotypes include, for example, AAV1, AAV2, AAV3, AAV4, AAV5, AAV6, AAV7, AAV8, AAV9, AAV10 and AAV11.

Vectors derived from AAV are particularly attractive for delivering genetic material because they are able to infect (transduce) a wide variety of non-dividing and dividing cell types including muscle fibers and neurons and they are devoid of the virus structural genes, thereby eliminating the natural host cell responses to virus infection, e.g., interferon-mediated responses. In addition, wild-type viruses have never been associated with any pathology in humans.

According to the present invention scAAV are also within the group of AAVs. Self-complementary adeno-associated vectors (scAAV) are viral vectors engineered from the naturally occurring adeno-associated virus (AAV) for use in gene therapy. ScAAV is termed “self-complementary” because the coding region has been designed to form an intramolecular double-stranded DNA template.

Thus, in some embodiments, by an “AAV” is meant a vector or virus derived from an adeno-associated virus serotype, including without limitation, AAV-1, AAV-2, AAV-3, AAV-4, AAV-5, AAV-6, AAV-7, AAV-8, AAV-9, AAV-10 and AAV-11. AAV vectors can have one or more of the AAV wild-type genes deleted in whole or part, e.g., the rep and/or cap genes, but retain functional flanking ITR sequences. Functional ITR sequences are necessary for the rescue, replication and packaging of the AAV virion. Thus, an AAV vector is defined herein to include at least those sequences that provide for replication and packaging (e.g., functional ITRs) of the virus. The ITRs need not be the wild-type nucleotide sequences, and may be altered, e.g., by the insertion, deletion or substitution of nucleotides, so long as the sequences provide for functional rescue, replication and packaging. In one embodiment, the vector is an AAV-9 vector, with AAV-2 derived ITRs. Also by an “AAV” is meant the protein shell or capsid, which provides an efficient vehicle for delivery of vector nucleic acid to the nucleus of target cells.

The term “AAV” as used herein is intended to also encompass recombinant AAV.

Adeno-associated virus (AAV) are herein also called viruses, viral particles or viral vectors.

As used herein, the term “cell” or “cell line” refers to a single cell or to a population of cells capable of continuous or prolonged growth and division in vitro. In some embodiments, e.g. the terms “HEK293 cells”, “293 cells” or their grammatical equivalents are used interchangeably here and refer to the host/packing cell line used in the methods disclosed herein.

Suitable cells and cell lines have been described for use in production of AAVs and AdVs. The cells themselves may be selected from any biological organism, including prokaryotic (e.g., bacterial) cells and eukaryotic cells including insect cells, yeast cells and mammalian cells. Particularly desirable host cells are selected from among any mammalian species, including, without limitation, A549, WEHI, 3T3, 10T1/2, BHK, MDCK, COS 1, COS 7, BSC 1, BSC 40, BMT 10, VERO, WI38, HeLa, a HEK 293 cell, Saos, C2C12, L cells, HT1080, HepG2 and primary fibroblast, hepatocyte and myoblast cells derived from mammals including human, monkey, mouse, rat, rabbit, and hamster.

Generally, the expression cassette is composed of, at a minimum, a 5′ AAV inverted terminal repeat (ITR), a nucleic acid sequence encoding a desirable therapeutic, immunogen, or antigen operably linked to regulatory sequences which direct expression thereof, and a 3′ AAV ITR. In one embodiment, the 5′ and/or 3′ ITRs of AAV serotype 2 are used. However, 5′ and 3′ ITRs from other suitable sources may be selected. It is this expression cassette that is packaged into capsid proteins to form an AAV virus or particle.

In addition to the expression cassette, the cells contain the sequences which drive expression of AAVs in the cells (cap sequences) and rep sequences of the same source as the source of the AAV ITRs found in the expression cassette, or a cross-complementing source. The AAV cap and rep sequences may be independently selected from different AAV parental sequences and be introduced into the host cell in a suitable manner known to one in the art. While the full-length rep gene may be utilized, it has been found that smaller fragments thereof, i.e., the rep78/68 and the rep52/40 are sufficient to permit replication and packaging of the AAV.

The cells also require helper functions in order to package the AAV of the invention. Optionally, these helper functions may be supplied by a herpesvirus. In another embodiment, the necessary helper functions are each provided from a human or non-human primate adenovirus source, such as are available from a variety of sources, including the American Type Culture Collection (ATCC), Manassas, Va. (US).

During the manufacturing of AAVs, a percentage of capsids might not incorporate any of the transgenes and are referred to as empty capsids, empty AAVs or empty AAV particle. Additionally, capsids that contain fragments of the transgene are called partial capsids, partial AAVs or partial AAV particles. These undesired product-related impurities are co-produced with the full capsids or full AAVs which contain the full length of the desired transgene.

Purification means to increase the degree of purity of a target molecule, in this case the AAVs, e.g. by removing one or more impurities.

The term “impurity” or “contaminant” as used herein, refers to any foreign or objectionable molecules or species, including a biological macromolecules such as DNA, RNA, one or more host cell proteins, nucleic acids, endotoxins, lipids, impurities of synthetic origin like detergents, partial and/or empty AAVs or AdVs as well as one or more additives which may be present in a sample containing the viral particles to be purified and thus to be separated from one or more of the impurities.

As used herein, and unless stated otherwise, the term “sample” refers to any composition or mixture that contains AAVs. Samples may be derived from biological or other sources. Biological sources include eukaryotic and prokaryotic sources, such as plant and animal cells, tissues and organs. The sample may also include diluents, buffers, detergents, and contaminating species, debris and the like that are found mixed with the target molecule. The sample may be “partially purified” (i.e., having been subjected to one or more purification steps, such as filtration steps) or may be obtained directly from a host cell producing the AAVs, e.g., the sample may comprise harvested cell culture fluid.

Alkyldimethylamine oxides suitable for lysing the virus producing cells are amphipathic, charged amine oxides coupled to saturated hydrocarbon chains of varying lengths. Preferably, the length of the saturated hydrocarbon chain is between 8 and 18 carbon atoms. In a preferred embodiment, the alkyldimethylamine oxides are selected from the group consisting of dimethyldecylamineoxide, dimethylundecylamineoxide, dimethyldodecylamineoxide (LDAO), dimethyltridecylamineoxide and dimethytetradecylamineoxide (TDAO), especially preferred is TDAO.

These compounds are preferably used at concentrations above their critical micelle concentration. The critical micelle concentration (CMC) is defined as the concentration of detergents above which micelles form and all additional detergents added to the system go to micelles. The value of the CMC for a given detergent in a given medium depends on temperature, pressure, and (sometimes strongly) on the presence and concentration of other surface active substances and electrolytes.

The terms “purifying,” “separating,” or “isolating,” as used interchangeably herein, refer to increasing the degree of purity of the target AAVs from a composition or sample comprising the target AAVs and one or more impurities.

The term “chromatography” refers to any kind of technique which separates an analyte of interest (e.g. a target AAV) from other molecules present in a sample. Usually, the target AAV is separated from other molecules as a result of differences in rates at which the individual molecules of the mixture bind to and/or migrate through a chromatography matrix under the influence of a moving phase.

The term “matrix” or “chromatography matrix” are used interchangeably herein and refers to a solid phase through which the sample migrates in the course of a chromatographic separation. The matrix typically comprises a base material and ligands covalently bound to the base material. The matrix of the present invention comprises or consists e.g. of particles, a membrane or a monolith, preferably the base material is a membrane or monolith, most preferred a membrane.

A “ligand” is a functional group that is part of the chromatography matrix, typically it is attached to the base material of the matrix, and that determines the binding properties and interaction properties of the matrix. Examples of “ligands” include, but are not limited to, ion exchange groups, hydrophobic interaction groups, hydrophilic interaction groups, thiophilic interactions groups, metal affinity groups, affinity groups, bioaffinity groups, and mixed mode groups (combinations of the aforementioned). It is also possible that one ligand has more than one binding/interaction property.

The matrix of the present invention comprises at least cation exchange groups and hydrophobic interaction groups. It is a mixed mode cation exchange chromatography matrix. The cation exchange groups may for example be strong cation exchange groups, like sulfonic acid groups. They may also be weak cation exchange groups, such as carboxymethyl or carboxylic acids. Examples of hydrophobic interaction groups are hydrophobic groups like phenyl, butyl, propyl, hexyl. The matrix may comprise two or more different cation exchange groups, e.g. weak and strong cation exchange groups. It may also comprise two or more different hydrophobic interaction groups. It may additionally comprise further other types of ligands. The groups may be part of the base material, they may also be part of a ligand. One ligand may comprise one or several different cation exchange and/or hydrophobic groups.

The ligands can be attached to the base material of the matrix by any type of covalent attachment. Covalent attachment can for example be performed by directly bonding the functional groups to suitable residues on the base material like OH, NH2, carboxyl, phenol, anhydride, aldehyde, epoxide or thiol etc. It is also possible to attach the ligands via suitable linkers. It is also possible to generate the matrix by polymerizing monomers comprising the ligands and a polymerizable moiety. Examples of matrices generated by polymerization of suitable monomers are polystyrene, polymethacrylamide or polyacrylamide based matrices generated by polymerizing suitable styrole or acryloyl monomers.

In another embodiment the chromatography matrix can be generated by grafting the ligands onto the base material or from the base material. For grafting from processes with controlled free-radical polymerisation, such as, for example, the method of atom-transfer free-radical polymerisation (ATRP), are suitable. A very preferred one-step grafting from polymerisation reaction of acrylamides, methacrylates, acrylates, methacrylates etc. which are functionalized e.g. with ionic, hydrophilic or hydrophobic groups can be initiated by cerium (IV) on a hydroxyl-containing support, without the support having to be activated.

When the chromatography matrix is used in a chromatographic separation it is typically used in a separation device, also called housing, as a means for holding the matrix.

In one embodiment, the device comprises a housing with an inlet and an outlet and a fluid path between the inlet and the outlet. In one embodiment the device is a chromatography column. Chromatography columns are known to a person skilled in the art. They typically comprise cylindrical tubes or cartridges filled with the stationary phase as well as filters and/or means for fixing the stationary phase in the tube or cartridge and optionally connections for solvent delivery to and from the tube or cartridge. The size of the chromatography column varies depending on the application, e.g. analytical or preparative. In one embodiment the column or generally the separation device is a single use device.

In certain embodiments, the housing comprises
a housing unit, wherein the housing unit comprises
(a) an inlet and an outlet;
(b) a fluid flow path between the inlet and the outlet; and
(c) a chromatography matrix within the housing unit.

In one embodiment the chromatography matrix comprises
a support member comprising a plurality of pores extending through the support member; and
a non-self-supporting macroporous cross-linked gel comprising macropores having an average size of 10 nm to 3000 nm, said macroporous gel being located in the pores of the support member;
wherein said macropores of said macroporous cross-linked gel are smaller than said pores of said support member;
wherein the pores of the support member are substantially perpendicular to the fluid flow path.

In certain embodiments, the invention relates to a fluid treatment device comprising
a plurality of housings, especially housing units, wherein each housing unit comprises
(a) an inlet and an outlet;
(b) a fluid flow path between the inlet and the outlet; and
(c) a chromatography matrix within the housing unit

In certain embodiments, especially if the chromatography matrix is a membrane, the chromatography matrix is arranged in the housing in a substantially coplanar stack of substantially coextensive sheets, a substantially tubular configuration, or a substantially spiral wound configuration.

A “buffer” is a solution that resists changes in pH by the action of its acid-base conjugate components. Various buffers which can be employed depending, for example, on the desired pH of the buffer are described in Buffers. A Guide for the Preparation and Use of Buffers in Biological Systems, Gueffroy, D., ed. Calbiochem Corporation (1975). Non-limiting examples of buffers include MES, MOPS, MOPSO, Tris, HEPES, phosphate, acetate, citrate, succinate, and ammonium buffers, as well as combinations of these.

According to the present invention the term “buffer” or “solvent” is used for any liquid composition that is used to load, wash, elute, re-equilibrate, strip and/or sanitize the chromatography matrix.

When “loading” a chromatography column in bind and elute mode, the sample or composition comprising the target molecule and one or more impurities is loaded onto a chromatography column. In preparative chromatography, the sample is preferably loaded directly without the addition of a loading buffer. If a loading buffer is used, the buffer has a composition, a conductivity and/or pH such that the target AAV is bound to the stationary phase while ideally all the impurities like the host cell proteins or empty AAVs are not bound and flow through the column. Typically, the loading buffer, if used, has the same or similar composition as the equilibration buffer used to prepare the column for loading. The final composition of the sample loaded on the column is called feed. The feed may comprise the sample and the loading buffer but preferably it is only the sample.

By “wash” or “washing” a chromatography matrix is meant passing an appropriate liquid, e.g. a buffer through or over the matrix. Typically washing is used to remove weakly bound impurities from the matrix in bind/elute mode prior to eluting the target molecule. Additionally, wash steps can be used to reduce levels of residual detergents, enhance viral clearance and/or alter the conductivity carryover during elution.

To “elute” a molecule (e.g. the target AAVs) from a matrix means that the molecule is removed therefrom. Elution may take place by altering the solution conditions such that a buffer different from the loading and/or washing buffer competes with the molecule of interest for the ligand sites on the matrix or alters the equilibrium of the target molecule between stationary and mobile phase such that it favors that the target molecule is preferentially present in elution buffer.

A non-limiting example is to elute a molecule from a mixed mode cation exchange resin by altering the ionic strength of the buffer surrounding the mixed mode cation exchange material such that the buffer competes with the molecule for the charged sites on the ion exchange material. Alternatively, a pH change is also suitable.

The terms “flow-through process”, “flow-through mode”, and “flow-through operation”, as used interchangeably herein, refer to a chromatographic process in which at least one target molecule (e.g. an AAV) contained in a sample along with one or more impurities is intended to flow through a chromatography matrix, which usually binds one or more impurities, where the target molecule usually does not bind (i.e., flows through) and is eluted from the chromatograph matrix with the loading buffer.

The terms “bind and elute mode” and “bind and elute process”, as used herein, refer to a separation technique in which at least one target molecule contained in a sample (e.g. an AAV) binds to a suitable chromatography matrix and is subsequently eluted with a buffer different from the loading buffer.

The term “ionic density” as used herein, refers to number of ions per unit of volume or mass of a given separation material, more particularly, the number of ions of given type (e.g. positive ions or negative ions) per unit volume or mass of separation material. Usually the number of ions is estimated titrating the given separation material. Moreover, the amount of the ions is given in equivalents (eq) per mass or volume unit for separation material.

The term “conductivity” as used herein, refers to an inherent property of most materials, that quantifies how strongly it resists or conducts electric current. In aqueous solutions, such as buffers, the electrical current is carried by charged ions. The conductivity is determined by the number of charged ions, the amount of charge they carry and how fast they move. Hence, for most aqueous solutions, the higher the concentration of dissolved salts, the higher the conductivity. Raising the temperature enables the ions to move faster, hence increasing the conductivity. Typically, the conductivity is defined in room temperature, if not otherwise indicated. The basic unit of conductance is Siemens(S). It is defined as the reciprocal of the resistance in Ohms, measured between the opposing faces of a 1 cm cube of liquid. Therefore, the values are estimated in S/cm.

Log reduction is a measure of how thoroughly a purification process reduces the concentration of a contaminant. It is defined as the common logarithm of the ratio of the levels of contamination before and after the process, so an increment of 1 corresponds to a reduction in concentration by a factor of 10. In general, an n-log reduction means that the concentration of remaining contaminants is only 10-n times that of the original. So for example, a 0-log reduction is no reduction at all, while a 1-log reduction corresponds to a reduction of 90 percent from the original concentration, and a 2-log reduction corresponds to a reduction of 99 percent from the original concentration.

A membrane as chromatographic matrix can be distinguished from particle-based chromatography by the fact that the interaction between a solute, e.g. the target AAVs or contaminants, and the matrix does not take place in the dead-ended pores of a particle, but mainly in the throughpores of the membrane. Exemplary types of membranes are flat sheet systems, stacks of membranes, microporous polymer sheets with incorporated cellulose, polystyrene or silica-based membranes as well as radial flow cartridges, hollow fiber modules and hydrogel membranes. Preferred are hydrogel membranes. Such membranes comprise a membrane support and a hydrogel formed within the pores of said support. The membrane support provides mechanical strength to the hydrogel. The hydrogel determines the properties of the final product, like pore size and binding chemistry.

The membrane support can consist of any porous membrane like polymeric membranes, ceramic based membranes and woven or non-woven fibrous material. Suitable polymeric materials for membrane supports are cellulose or cellulose derivatives as well as other preferably inert polymers like polyethylene, polypropylene, polybutylenterephthalate or polyvinylidene-difluoride.

The hydrogels can be formed through in-situ reaction of one or more polymerizable monomers with one or more crosslinkers and/or one or more cross-linkable polymers to form a cross-linked gel that has preferably macropores. Suitable polymerizable monomers include monomers containing vinyl or acryl groups. Preferred are monomers comprising an additional functional group that either directly forms the ligand of the matrix or is suitable for attaching the ligands. Suitable crosslinkers are compounds containing at least two vinyl or acryl groups. Further details about suitable membrane supports, monomers, crosslinkers etc. as well as suitable production conditions can be found in WO04073843 and WO2010/027955. Especially preferred are membranes made of an inert, flexible fiber web support comprising assembly within and around the fiber web support a porous polyacrylamide hydrogel with cation exchange as well as hydrophobic groups, like Natrix@ type membranes, Merck KGaA, Germany. Further details about suitable hydrogel mixed mode membranes can be found in WO2014018635.

Examples of suitable membranes to be used in the method of the present invention are
Membranes with a polyethersulfone (PES)-based support and a cross-linked polymeric coating, functionalized with suitable ligands, like Mustang® type membranes, Pall.
Membranes made of stabilized reinforced cellulose, functionalized with suitable ligands, like Sartobind® type membranes, Sartorius.
Membranes made of stabilized reinforced cellulose, comprising a hydrogel with suitable ligands, like Sartobind® Jumbo Membranes, Sartorius, made of stabilized reinforced cellulose
Membranes made of a fine fiber non-woven scaffold comprising a hydrogel with suitable ligands, like 3M™ Emphaze™ Hybrid Purifier type membranes, 3M.
Membranes made of an inert, flexible fiber web support comprising within and around the fiber web support a porous polyacrylamide hydrogel with suitable cation exchange and hydrophobic ligands, like Natrix@ type Chromatography membranes, Merck KGaA, Germany.

A monolith or a monolithic sorbent, similar to a membrane, has throughpores, like interconnected channels, so that liquid can flow from one side of the monolith, through the monolith, to the other side of the monolith.

Since the mobile phase is flowing through these throughpores, molecules to be separated are transported by convection rather than by diffusion. Due to their structure monolithic sorbents show flow rate independent separation efficiency and dynamic capacity.

The monolith is typically formed in situ from reactant solutions and can have any shape or confined geometry, typically with frit-free construction, which guarantees convenience of operation. Preferably, monolithic materials have a binary porous structure, mesopores and macropores. The micron-sized macropores are the throughpores and ensure fast dynamic transport and low backpressure in applications; mesopores contribute to sufficient surface area and thus high loading capacity.

The monoliths can be made of organic, inorganic or organic/inorganic hybrid materials. Preferred are organic polymer based monoliths.

The synthesis of organic polymer monoliths is typically done by a one-step polymerization providing a tunable porous structure with tailored functional groups. Generally, a pre-polymerization mixture consisting of the monomers, crosslinkers, porogenic solvents, and initiators in an appropriate ratio is polymerized in a suitable container, also called mould, determining the format of the monolith. Polymerization is typically initiated by heating, use of UV radiation, microwave or γ-ray radiation in the presence of initiators. After reaction for the prescribed time at an appropriate temperature, the resulting material is typically washed with solvents to remove unreacted components and porogenic solvents.

Suitable organic polymers are polymethacrylates, polyacrylamides, polystyrenes, polyurethanes, etc., like Poly(methacrylic acid-ethylene dimethacrylate), Poly(glycidyl methacrylate-ethylene dimethacrylate) or Poly(acrylamide-vinylpyridine-N,N′-methylene bisacrylamide).

Inorganic monoliths can be made of silica or other inorganic oxides. Preferably they are made of silica. Silica monoliths are normally prepared via a sol-gel method with phase separation. This mainly includes hydrolysis, condensation, and polycondensation of silica precursors. Typically, tetraethoxysilane (TEOS) or tetramethylorthosilicate (TMOS) is distributed in a suitable solvent in the presence of a porogen (e.g. poly(ethylene glycol) (PEG)), followed by the addition of a catalyst, acid or base, or a binary catalyst, acid and base in sequence. After reaction for a prescribed time, the resulting gel-like product is washed with solvents to remove unreacted precursor, porogen, and catalyst, followed by the proper post treatment, typically a heat treatment.

The monoliths can be modified with suitable functional groups, in the present case cation exchange groups and hydrophobic interaction groups, to generate the targeted interaction with the sample comprising the target molecule and thus the targeted separation.

Typically the monoliths are contained in a housing like a column.

Membranes and monoliths can also be produced by 3D printing processes.

Particulate base materials can be prepared, for example, from organic polymers. Organic polymers of this type can be polysaccharides, such as agarose, dextranes, starch, cellulose, etc., or synthetic polymers, such as poly(acrylamides), poly(methacrylamides), poly(acrylates), poly(methacrylates), hydrophilically substituted poly(alkyl allyl ethers), hydrophilically substituted poly(alkyl vinyl ethers), poly(vinyl alcohols), poly(styrenes) and copolymers of the corresponding monomers. These organic polymers can preferably also be employed in the form of a crosslinked hydrophilic network. This also includes polymers made from styrene and divinylbenzene, which can preferably be employed, like other hydrophobic polymers, in a hydrophilized form.

Alternatively, inorganic materials, such as silica, zirconium oxide, titanium dioxide, aluminium oxide, etc., can be employed as particulate base materials. It is equally possible to employ composite materials, i.e., for example, particles which can themselves be magnetised by copolymerisation of magnetisable particles or of a magnetisable core. It is also possible to use core shell materials whereby the shell, i.e. at least the surface or a coating, has OH groups.

However, preference is given to the use of hydrophilic base materials which are stable to hydrolysis or can only be hydrolysed with difficulty since the materials according to the invention should preferably withstand alkaline cleaning or regeneration at e.g. basic pH over an extended use duration.

The base matrix may consist of irregularly shaped or spherical particles, whose particle size can be between 2 and 1000 μm. Preference is given to average particle sizes between 3 and 300 μm, in a most preferred embodiment the average particle size is between 20-63 μm.

The particulate base material may, in particular, be in the form of non-porous or preferably porous particles. The average pore sizes can be between 2 and 300 nm. Preference is given to pore sizes between 5 and 200 nm, most preferred average pore size is between 40-110 nm.

In a very preferred embodiment, the particulate base material is formed by copolymerisation of a hydrophilically substituted alkyl vinyl ether selected from the group of 1,4-butanediol monovinyl ether, 1,5-pentanediol monovinyl ether, diethylene glycol monovinyl ether or cyclo-hexane-dimethanol monovinyl ether and divinylethyleneurea (1,3-divinylimidazolin-2-one) as crosslinking agent.

An example of a suitable commercially available vinylether based base material is Eshmuno@, Merck KGaA, Germany.

Preferred particulate matrices are matrices with weak cation exchange, strong cation exchange and hydrophobic groups, e.g. with sulfonic acid, carboxylic acid and phenyl groups, like Eshmuno® HCX, Merck KGaA, Germany. Also preferred is a particulate chromatography matrix comprising of a hydroxyl group containing base material, preferably a vinylether based base material, to the surfaces of which polymer chains are grafted by covalent bonding, characterised in that
a) the polymer chains are covalently bonded to the base matrix via the hydroxyl groups,
b) the polymer chains comprise end groups-N(Y)—R3 with Y being independently from each other H or CH₃, preferably H, and R3 being —CHCOOMR4
with R4 being C1 to C4 alkyl, like methyl, ethyl, propyl, isopropyl, butyl, isobutyl, preferably isopropyl and isobutyl, very preferred isobutyl, or C1 to C4 perfluoroalkyl
and M being independently from each other H, Na, K, or NH₄_{⁺}.

An example is Eshmuno® CMX, Merck KGaA,

The base material may equally also be in the form of fibres, hollow fibres or coatings.

The present invention provides methods of separating or purifying AAVs. This means that one or more AAVs can be separated from one or more other AAVs, from empty or partial AAVs and/or from other impurities in a sample. Preferably at least one AAV is separated from at least one impurity. This is done by a chromatographic separation on a mixed mode cation exchange hydrophobic chromatography matrix which comprises at least one type of cation exchange groups and one type of hydrophobic groups.

By the methods of the invention AAVs can be separated, enriched and/or purified enabling an efficient separation. In particular aspects of the invention, target AAVs can be separated from impurities and empty AAVs in one chromatographic step. A high-resolution separation can be achieved and different rAAV species can be isolated.

The production of cells comprising AAVs is known to a person skilled in the art. Typically, the selected cells are expanded in suitable culture media in a bioreactor under suitable conditions. The cells may be grown as adherent or suspension culture. For example, in suspension culture of HEK293 cells suitable seeding numbers before transfection are 0.5 to 1.1 e6 viable cells per ml.

Suitable methods for the transduction are known in the art. In one embodiment, cells can be transduced in vitro by combining a rAAV with the cells, e.g., in appropriate media, and screening for those cells harboring the DNA of interest using conventional techniques such as Southern blots and/or PCR, or by using selectable markers.

Transfection can be performed using any of the techniques known in the art, including but not limited to electroporation, lipofection, e.g. with a lipofectamine, cationic polymers and cationic lipids. Any suitable transfection media may be used. In one embodiment of the transfection process, adherent or suspension human embryonic kidney (HEK293) cells are transfected with a triple DNA plasmid polyethylenimine (PEI) co-precipitation.

In an embodiment, the present disclosure provides a method for manufacturing an AAV based viral vector comprising the steps of (i) culturing cells in a bioreactor or flask, (ii) transfecting the cells with plasmids to enable production of the AAV particles, (iii) lysing the mixture of the cells and the viral particles to release of the viral particles from the cells iv) isolating and/or purifying the viral particles whereby step iv) comprises a chromatographic purification on a mixed mode cation exchange chromatography matrix.

After a suitable virus production period post transfection or infection, the cells are lysed and the viral particles harvested. In some embodiments, the cells are dissociated from the bioreactor before the cell lysis process is initiated. In some embodiments, the cells are lysed in situ.

Preferably, for lysis, the cells are contacted with a composition comprising an alkyldimethylamine oxide and optionally sodium chloride. Preferably, the lysis solution is added to the bioreactor comprising the suspension of the cells so that a mixture of the cell suspension and the composition is generated.

Incubation of the mixture comprising the cells and the composition comprising an alkyldimethylamine oxide and optionally sodium chloride is done typically for 30 to 180 minutes, preferably for an incubation time between 60 and 90 minutes. Shorter and longer times may also be appropriate.

The pH of the mixture during the incubation can vary in broad range. It can for example be between pH 4 and pH 10, typically it is between pH 6 and pH 9.

The temperature of the mixture during incubation can also vary in a broad range. It can for example be between 2° and 37° C.

The concentration of the alkyldimethylamine oxide in the composition is such that it effectively induces cell lysis, that means that after the incubation with the composition under suitable conditions as described above, at least 80%, preferably 100% of the cells are lysed. For this, the concentration of the alkyldimethylamine oxide in the final mixture with the cells is preferably above its CMC. For TDAO, N,N-Dimethyltridecylamine N-oxide, the concentration in the mixture with the cells is typically between 0.1% and 5% (w/w), preferably between 1% (w/w) and 4% (w/w). Typically the volume of the composition that is added is smaller than the volume of the cell culture.

Consequently, suitable concentrations of the alkyldimethylamine oxide, especially TDAO, in the composition are between 10 and 30% (w/w). Suitable concentrations of sodium chloride in the composition to be added to the cell suspension are between 1 and 6 mol/l, typically around 3 to 5 mol/l.

After incubation with the lysis composition the released AAVs can then be isolated and/or purified, whereby one chromatographic purification on a cation exchange mixed mode matrix is included.

Optionally, after lysis the obtained mixture is first filtered or centrifuged.

In one embodiment the mixture is filtered through a filter that removes large molecule contaminants and cellular debris but that permits AAVs to pass therethrough.

In one embodiment, the released viral particles can be separated and purified from the cell culture medium using clarification. Clarification can be a microfiltration process in which relatively larger components such as lysed cells and/or impurities are removed from a solution. Clarification filters include depth filtration, charged depth filtration and similar microfiltration techniques.

Tangential flow filtration can be used to concentrate the mixture of purified viral particles and to remove salts and proteins. Tangential flow filtration (TFF) refers to a generally rapid and efficient method for filtration or purification of a solution containing target product and/or impurities during which a solution or liquid stream flows parallel to a filtering membrane.

Centrifugation can for example be a low speed centrifugation to remove larger particles like cellular debris. This can be for example done at 10000 to 12000 g for 10 to 30 minutes. The released viral particles can be found in the supernatant.

The isolation and/or purification of the AAVs typically includes one or more of the following process steps:
Clarification
filtration
dialysis/diafiltration
tangential flow filtration
treatment with nuclease, e.g. RNase and/or DNase
treatment with chloroform
ion exchange chromatography
affinity chromatography
hydrophobic interaction chromatography
centrifugation
PEG precipitation

In some embodiments, a nuclease, typically an endonuclease, is added, e.g. to reduce the amount of host cell DNA. It can be added directly to the mixture in the bioreactor before, while or after lysis. The nuclease may be one that degrades both DNA and RNA. In one embodiment, the endonuclease is a genetically engineered endonuclease from Serratia marcescens that is sold under the name Benzonase® (EMD Millipore, US).

In the cation exchange mixed chromatography step, the target AAVs are separated from at least one impurity in a sample, whereby the sample comprising the AAVs is brought into contact with the chromatography matrix. Contact times are usually in the range from 6 seconds to 24 hours. It is advantageous to work in accordance with the principles of liquid chromatography by passing the liquid through a chromatography column or other type of housing which contains the mixed mode cation exchange chromatography matrix. The liquid can run through the column or housing merely through its gravitational force or be pumped through by means of a pump. An alternative method is batch chromatography, in which the separation material is mixed with the liquid by stirring or shaking for as long as the AAVs need to be able to bind to the separation material. It is likewise possible to work in accordance with the principles of the chromatographic fluidised bed by introducing the liquid to be separated into, for example, a suspension comprising the chromatography matrix, where the chromatography matrix is selected so that it is suitable for the desired separation owing to its high density and/or a magnetic core.

If the chromatographic process is run in the bind and elute mode, the target AAVs bind to the chromatography matrix. The chromatography matrix can subsequently be washed with one or more wash buffers, which preferably have the same ion strength and the same pH as the liquid in which the target molecule is brought into contact with the chromatography matrix. The wash buffer removes substances which do not bind to the chromatography matrix. Further washing steps with other suitable buffers may follow without desorbing the target AAVs. The desorption of the bound AAVs is carried out by changing the ion strength in the eluent and/or by changing the pH in the eluent and/or by changing the solvents. The target AAVs can thus be obtained in a purified and concentrated form in the eluent. The target AAVs usually have a purity above 70%, e.g. of 70 percent to 99 percent, preferably above 85%, e.g. 85 percent to 99 percent, particularly preferably above 90%, e.g. 90 percent to 99 percent, after desorption.

However, if the chromatographic process is run in the flow-through mode, the target AAVs remain in the liquid, but other accompanying substances bind to the separation material. The target AAV is then obtained directly by collecting the column eluate in through-flow. It is known to the person skilled in the art how he has to adapt the conditions, in particular the pH and/or the conductivity, in order to bind a specific biopolymer to a separation material, or whether it is advantageous for the purification task not to bind the target AAVs.

The cation exchange mixed mode material can be used for the purification of AAVs enabling an efficient removal of impurities like host cell proteins. Moreover, preferably, the same chromatography step additionally provides for removal of empty particles. Unexpectedly, it has been found, that empty particles elute together with host cell proteins from the cation exchange hydrophobic chromatography matrix under the preferred elution conditions at pH 4.5 to pH 7.8 and 13 to 20 mS/cm and can be separated by peak fractionation while the target AAVs are still bound to the chromatography matrix. In a preferred embodiment, the chromatography matrix is a membrane, especially a hydrogel membrane.

The nature of the chromatography matrix used (i.e. strong and/or weak cation exchangers, type of hydrophobic group) and the conditions of salt concentration, buffer used, and pH, will vary on the AAV capsid variant (i.e. AAV capsid serotype or pseudotype). While the known AAV capsid variants all share features such as size and shape, they differ in fine details of molecular topology and surface charge distribution. Hence, while all capsid variants are expected to be amenable to purification by mixed mode cation exchange chromatography optimal methods can be determined in a systematic manner using chromatography resin and buffer screening experiments, different conditions will be required for each AAV capsid variant to achieve efficient AAV particle purification. The determination of such conditions is readily apparent to the skilled artisan.

Preferably, the chromatographic purification is performed by using a pH change from 4-6.5 to a pH above 9, preferably between 9 and 11, most preferred to around pH 10 in gradient or step mode while the conductivity is kept at a constant level in the range of 13 to 20 mS/cm. In case of using a pH gradient, the sample that is applied to the chromatograph matrix is adjusted to the pH with which the pH gradient is started.

In general, every buffer substance known to a skilled person in art could be used to generate pH and conductivity stable aqueous solutions. Examples of suitable buffers to be used, are listed in Table 1 for equilibration and in Table 2 for elution. If pH and conductivity needs to be adjusted, concentrations of buffers might change.

TABLE 1
Chemicals used for preparation of
equilibration buffer at various pH

| Chemical | M (g/mol) | C (mol/L) |
|---|---|---|
| Citric acid | 210.14 | 0.0080 |
| Sodium dihydrogen phosphate | 137.99 | 0.0091 |
| monohydrate | | |
| Glycine | 75.06 | 0.0215 |
| Tris(hydroxymethyl)aminomethan | 121.14 | 0.0107 |
| Succinate | 162.05 | 0.0077 |
| Sodiumchlorid | 58.99 | 0.195 |
| NaOH 32% | 39.997 | 0.0111 |
| Acetic Acid Glacial | 60.050 | 0.025 |
| Monosodium phosphate | 137.990 | 0.025 |
| Sodium Acetate | 60.050 | 0.025 |
| Disodium hydrogen phosphate | 141.96 | 0.025 |
| anhydrous | | |
| MES × H2O | 195.2 | 0.01 |

TABLE 2
Chemicals used for preparation of elution buffer at various pH

| Chemical | M (g/mol) | C (mol/L) |
|---|---|---|
| Phosphoric acid | 97.994 | 0.022 |
| HEPES | 238.3012 | 0.074 |
| TAPS | 243.27 | 0.063 |
| CAPSO | 221.32 | 0.042 |
| Sodium hydroxide | 39.997 | 0.060 |
| Sodium Acetate | 60.050 | 0.025 |
| Disodium hydrogen phosphate | 141.96 | 0.025 |
| anhydrous | | |
| Sodium chloride | 58.440 | 0.500 |
| Acetic Acid Glacial | 60.050 | 0.025 |
| Monosodium phosphate | 137.990 | 0.025 |
| Sodium chloride | 58.440 | 0.500 |

It is also possible to perform elution on the chromatographic matrix by using a linear or step mode salt gradient. The salt may be selected from the group consisting of: NaCl, KCl, sulfate, formate and acetate; preferably NaCl. Typically, the gradient starts with a low salt concentration e.g. between 10 and 150 mM of salt, which is then increased until elution of the target AAV, e.g. until 150 to 1000 mM of salt.

Alternatively, pH and salt gradient can be combined.

The term “column volume” refers to the volume inside of a packed column not occupied by the chromatography matrix. This volume includes both the interstitial volume (volume outside of the matrix) and the own internal porosity (pore volume) of the matrix.

In a preferred embodiment the applied linear gradient lasts around 30 to 50 column volume (CV) plus an additional hold step at target elution buffer for at least 20 CV.

In another preferred embodiment the target AAV product recovery rates are above 30%, preferably above 60%.

Additionally, the application is not limited to bind and elute applications, but can be used in a flow-through mode. This is especially suitable for the separation of different types of AAVs, whereby the target AAVs remain in the flow through and other types of AAVs are bound to the chromatography matrix.

Further, the present invention provides a chromatography based AAV purification step which can be regenerated and is applicable in a wide operation window, e.g. pH 3-10; conductivity 1 mS/cm-50 mS/cm. In a preferred embodiment, the method of the present invention is used to purify samples comprising of a broad range of viral particle concentration e.g. in the range 1e11 viral particles of AAV per ml (1e11 vp/mL) samples to 2.5e13 vp/mL samples.

The pH window span is between pH 4.5 to pH 11, where in the more preferred embodiment is conductivity range between pH 5.3 to pH 10. The conductivity window span is between 1 mS/cm-50 mS/cm, where in the more preferred embodiment is conductivity range between 2-30 mS/cm.

The method of the present invention can be applied for all rAAV and wt serotypes without a need for previous process steps. Preferably, crude lysate (nuclease treated and clarified) can be applied directly to the chromatography matrix. The production of a crude lysate of the AAVs typically includes one or more of the following process steps which are performed after cell lysis:
Clarification
filtration
dialysis/diafiltration
tangential flow filtration
treatment with nuclease, e.g. RNase and/or DNase

A crude lysate has not been treated with any other chromatography and/or ultra-/centrifugation step.

In a very preferred embodiment, a sample, e.g. in form of a crude lysate which has been treated with a nuclease like Benzonase® (Merck GaA, Germany) and has been clarified, is applied to a membrane with cation exchange and hydrophobic groups at a pH between 4.5 and 6.5 and a conductivity around 14 to 19 mS/cm. Optionally the loaded membrane is washed with Equilibration buffer with a pH from 4.5 to 6.5 and a conductivity around 14 to 19 mS/cm.

Elution is performed by applying a linear pH gradient from a pH between 4.5 and 6.5 depending on the sample pH to a pH between 9.5 and 10.5 at constant conductivity below 20 mS/cm.

Elution preferably results in two fractions depending on the pH reached during this step. Fraction 1 elutes from the matrix in the pH range between 5.5 to 7.8 and comprises the majority of co-eluting HCPs and mainly empty rAAV particles. Fraction 2 elutes from the matrix in the pH range between 8.7 to 10.1 and comprises a highly enriched portion of full rAAV and an HCP log reduction of 3 log.

The preferred buffer composition is a buffer with a wide pH range from 4 to 11, e.g., but not limited to, the Good buffer system containing tris, phosphate and acetate as well as amino acids.

With the method of the present invention, AAVs can be purified efficiently. Cation exchange mixed-mode chromatography can be used as an orthogonal method to capture rAAV from crude lysate with very high recoveries in the eluate and independent of the serotype and expression host. Typically, contaminants can be reduced with a log reduction of 2 log or more. Preferably, the method achieves a host cell protein reduction of more than 2 log. The additional reduction or removal of empty particles provides for reduction of the overall process steps as no separate, additional process step is needed for this. Process- and product-related impurities can be removed in one single step.

The enrichment of full AAV capsids can be calculated as quotient of genome or DNA containing particle amount divided by total viral particle amount. In the enriched elution fractions generated with the method of the present invention this quotient reaches 1 which means that all present AAVs contain DNA and so are considered as 100% full particles. In not enriched fractions this quotient is typically below 0.5. In crude lysate this quotient is typically below 0.2. This shows that the purity of crude lysate was increased by at least 80% through a single purification step with a cationic and hydrophobic interaction matrix capture and pH elution. Especially the use of membrane based matrices provides for effective and fast purification. It is known to a skilled person in the art, that the purity increasement is highly dependent of the crude lysate quality. But even with a crude lysate as sample the method of the present invention provides for target AAV product recovery rates above 30%, preferably above 60%.

The present invention is further illustrated by the following figures and examples, however, without being restricted thereto.

The entire disclosure of all applications, patents, and publications cited above and below as well as the corresponding EP application EP22162928.0, filed Mar. 18, 2022, are hereby incorporated by reference.

### EXAMPLES

The following examples represent practical applications of the invention.

### Example 1

1 ml column of Eshmuno@ CMX was loaded with 7 mL of affinity purified rAAV2. The column was eluted with a pH Gradient from pH 4.5 to 9.0. The conductivity was kept constant at 400 mM NaCl.

TABLE 1
Buffer compostion of Equilibration buffer
A1. Adjusted to pH 4.5 with 1M HCl

| | M (g/mol) | C (mol/L) |
|---|---|---|
| Citric acid | 210.14 | 0.00796 |
| Sodium dihydrogen phosphate | 137.99 | 0.00907 |
| monohydrate | | |
| Glycine | 75.06 | 0.02154 |
| Tris(hydroxymethyl)aminomethan | 121.14 | 0.01067 |
| Succinate | 162.05 | 0.00767 |
| Sodiumchlorid | 58.99 | 0.4 |
| NaOH 32% | 39.997 | 0.01110 |

TABLE 2
Buffer composition of Elution buffer
B1. Adjusted to pH 9.0 with 1M NaOH

| | M (g/mol) | C (mol/L) |
|---|---|---|
| Citric acid | 210.14 | 0.00796 |
| Sodium dihydrogen phosphate | 137.99 | 0.00907 |
| monohydrate | | |
| Glycine | 75.06 | 0.02154 |
| Tris(hydroxymethyl)aminomethan | 121.14 | 0.01067 |
| Succinate | 162.05 | 0.00767 |
| Sodiumchloride | 58.99 | 0.4 |
| NaOH 32% | 39.997 | 0.04600 |

| Step | Column volumes/mL | Buffer |
|---|---|---|
| Equilibration | 2 CV | Equilibration buffer A1 |
| Load | 7.08 diluted sample | — |
| Wash | 5 CV | Equilibration buffer A1 |
| Elution | 50 CV, 0-100% B1 | Equilibration buffer A1/ |
| | followed by 5 CV 100% | elution buffer B1 |
| | B1 | |
| Strip | 5 CV | Strip buffer |
| Re- | 2 CV | Equilibration buffer A1 |
| equilibration | | |

FIG. 1 shows the run on Eshmuno@ CMX column, running on the ÄKTA™ system. During the load no AAV could be detected. In the wash step the first peak occurred and during the elution gradient a second peak could be eluted. The overall recovery of all rAAV2 capsids was 78% and 77% for full rAAV2 capsids, but no enrichment in the elution peak was observed.

The overall recovery of all rAAV2 capsids was 78% and 77% for full rAAV2 capsids, but no enrichment in the elution peak was observed.

### Example 2

In this case study, the critical experimental parameters considered are pH with a range from 4.5 to 6.5 and salt concentration, with a range from 20 mM NaCl to 120 mM NaCl, because it is more reliable than conductivity which strongly depends on the temperature. The focus was on binding condition, while the elution condition was fixed. The output value required are dynamic binding capacity, calculated as vp/mL of device, and elution yield, calculated as the ratio between the concentration in the load volume and the concentration in the elution volume

TABLE 3
Buffer A1

| | Molar mass | Concentration | Target weight |
|---|---|---|---|
| Chemical | (g/mol) | (mol/L) | for 2 L (g) |
| Acetic Acid | 60.050 | 0.025 | 3.003 |
| Glacial | | | |
| Monosodium | 137.990 | 0.025 | 6.900 |
| phosphate | | | |

TABLE 3
Buffer A2

| | Molar mass | Concentration | Target weight |
|---|---|---|---|
| Chemical | (g/mol) | (mol/L) | for 2 L (g) |
| Acetic Acid | 60.050 | 0.025 | 3.003 |
| Glacial | | | |
| Monosodium | 137.990 | 0.025 | 6.900 |
| phosphate | | | |
| Sodium | 58.440 | 0.500 | 58.440 |
| chloride | | | |

TABLE 4
Buffer B1

| | Molar mass | Concentration | Target weight |
|---|---|---|---|
| Chemical | (g/mol) | (mol/L) | for 2 L (g) |
| Sodium | 60.050 | 0.025 | 3.003 |
| Acetate | | | |
| Disodium | 141.96 | 0.025 | 7.098 |
| hydrogen | | | |
| phosphate | | | |
| anhydrous | | | |

TABLE 5
Buffer B2

| | Molar mass | Concentration | Target weight |
|---|---|---|---|
| Chemical | (g/mol) | (mol/L) | for 2 L (g) |
| Sodium | 60.050 | 0.025 | 3.003 |
| Acetate | | | |
| Disodium | 141.96 | 0.025 | 7.098 |
| hydrogen | | | |
| phosphate | | | |
| anhydrous | | | |
| Sodium | 58.440 | 0.500 | 58.440 |
| chloride | | | |

The second step is required to achieve the desired pH value, 4.5 (1), 5.5 (2) and 6.5 (3). For each pH value we prepared a salt-free buffer (a) and a 1 M NaCl buffer (b).

The membrane was firstly sanitized by flushing it with 1M NaOH solution and leaving the device in a static soak for 30 minutes. Then it was flushed with equilibration buffer until the desired condition were reached.

Before loading, it is necessary to adjust the pH and conductivity of the sample. Therefor the feed was diluted 1:10 with equilibration buffer per each explored condition.
Equilibration (25 mM sodium acetate, 25 mM phosphate, 120 mM NaCl, pH 5.3, 5 MV)
Load (AAV2 crude lysate, pH 5.3, 17 mS/cm). Total loaded volume˜640 mL, almost 2E+13 vp/mL device.
Wash (25 mM sodium acetate, 25 mM phosphate, 120 mM NaCl, pH 5.3, 20 MV)
Elution (25 mM sodium acetate, 25 mM phosphate, 500 mM NaCl, pH 8.5. 20 MV). Step gradient elution was applied.
Strip (0.1 M Tris, 2 M NaCl, pH 10.6. 20 MV)
CIP (1 M NaOH. 20 MV)
Re-equilibration B (25 mM sodium acetate, 25 mM phosphate, 500 mM NaCl, pH 8.5. 30 MV)
Re-equilibration A (25 mM sodium acetate, 25 mM phosphate, 120 mM NaCl, pH 5.3, 30 MV)

Fractionation was performed for flow-through, wash, elution and strip step, then each sample was stored at −80° C. to be further analyzed.

FIG. 2 shows the chromatogram of AAV2 of capture run using a step gradient elution on mixed-mode membrane of 1 mL volume. Equilibration buffer consists of a combination of salts and 120 mM NaCl and pH 5.3, whereby the elution buffer contains 500 mM NaCl and same constituting salts and pH 8.5.

### Example 3

To improve HCP removal in chromatographic process, pH variation has been investigated as improving parameter for cation exchange chromatography in the field of antibody purification for the HCP removal. Therefore, capture run with increased pH for loading buffer and same process steps applied for previous capture run has been carried out and the chromatogram is reported in FIG. 2:

FIG. 3 shows Chromatogram of AAV2 of capture run using a step gradient elution on mixed-mode membrane of 1 mL volume, aiming to improve HCP clearance. Operating conditions: pH 6.5, 120 mM NaCl, whereby the elution buffer contains 500 mM NaCl and same constituting salts and pH 8.5.

The HCPs amount assessed in the analyzed fractions confirms what was observed from silver stained gel, where AAV2-related impurities bands were detected in the flow-through fractions and especially in the elution fraction. The HCP level determined by ELISA resulted in a log reduction of 1.5 in the elution fraction, but this cannot be considered as a valuable HCP removal

### Example 4

1 mL Natrix® CH membrane device was loaded with 13 mL rAAV2 crude lysate of HEK293 cells (Benzonase@ treated and clarified; 1:10 diluted in equilibration buffer). The elution was performed under constant conductivity with 150 mM Na+ and with a linear pH gradient from pH 5.3 to 10.

FIG. 4 shows Chromatogram of rAAV2 capture run using a linear gradient elution on mixed-mode membrane of 1 mL volume, aiming to improve HCP clearance and full rAAV2 capsids on step. Binding conditions: pH 5.3, 150 mM NaCl, whereby the elution buffer contains 150 mM NaCl and same constituting salts and pH 10.

In this example the majority of the HCPs was found in the flow-through fraction 98%, 3% eluted in the first peaks together with 30% of empty rAAV2 capsids. In the second peak a highly pure rAAV2 fraction can be detected. The HCP log removal was almost 3 and 2 fractions contained nearly 70% of full rAAV2.

1. A method for purifying adeno-associated virus (AAV) particles by contacting a sample comprising said AAV particles with a mixed mode cation exchange chromatography matrix.

2. Method according to claim 2 whereby it comprises the steps of
a) contacting the sample comprising the AAV particles with a mixed mode cation exchange chromatography matrix
b) optionally washing the chromatography matrix
c) eluting AAV particles which bound to the chromatography matrix in step a) with an elution buffer

3. Method according to claim 1, whereby in step c) the elution buffer has a pH higher than the pH of the sample in step a).

4. Method according to claim 1, whereby the method includes recovering AAV particles which flow through the chromatography matrix without binding to it.

5. Method according to claim 1, whereby the chromatography matrix is a membrane or a monolith.

6. Method according to claim 1, whereby is a hydrogel membrane.

7. Method according to claim 1, whereby the sample comprises empty and full AAV capsids and the empty AAV particles are partially or fully eluted prior to the full AAV particles.

8. Method according to claim 1, whereby the sample is a crude lysate.

9. Method according to claim 1, whereby elution in step c) is performed with a linear pH gradient from a pH between 4 and 6.5 to a pH between 9 and 11 while conductivity is kept at a constant level.

10. Method according to claim 1, whereby in one fraction of AAVs eluted in step c) HCPs are reduced with a log reduction of 2 log or more compared to the sample applied in step a) and the percentage of full AAVs is above 90%.

11. A method for purifying AAV particles from a sample comprising cells enclosing the AAV particles by
a) lysing the cells
b) isolating and/or purifying the AAV particles by contacting it with a mixed mode cation exchange chromatography matrix according to the method of claim 1.

12. Method according to claim 8, whereby lysis of the cells is done with a detergent selected from the group of alkyldimethylamine oxides and optionally sodium chloride.

13. Method according to claim 11, whereby the method comprises one or more of the following steps:
Clarification
filtration
dialysis/diafiltration
tangential flow filtration
treatment with nuclease, e.g. RNase and/or DNase
treatment with chloroform
ion exchange chromatography
affinity chromatography
hydrophobic interaction chromatography
centrifugation
PEG precipitation
